(19) United States
(12) Patent Application Publication
(10) Pub. No.: US 2012/0321685 A1
(43) Pub. Date: Dec. 20, 2012

(54) COMPOSITIONS CONTAINING OILS HAVING A SPECIFIC GRAVITY HIGHER THAN THE SPECIFIC GRAVITY OF WATER

(75) Inventors: Noa LAPIDOT, Mevasseret Zion (IL); Claudio ROTTMAN, Modiin (IL); Ludmila SOSONKIN, Jerusalem (IL); Fabio BIAGINI, Bologna (IT); Oleg NAIGERTSIK, Rehovot (IL)

(73) Assignee: SOL-GEL TECHNOLOGIES LTD., Beit Shemesh (IL)

(21) Appl. No.: 13/597,219

(22) Filed: Aug. 28, 2012

Related U.S. Application Data
(63) Continuation of application No. 10/494,969, filed on Oct. 14, 2004, now abandoned.
(60) Provisional application No. 60/331,104, filed on Nov. 8, 2001.

(51) Int. Cl. A61K 8/11; A61Q 17/04; A61K 38/43; A01N 25/28; A61Q 19/02; A61Q 5/02; A61Q 19/10; A61Q 1/02; A61Q 1/06; A61P 17/00; A61P 31/04; A61P 31/10; A61P 17/06; A61P 1/02; A61K 9/50

(52) U.S. Cl. 424/401; 424/451; 424/59; 424/94.1; 424/408; 424/62; 514/169; 424/60; 510/119; 510/130; 424/63; 424/64

(57) ABSTRACT

The invention relates to pharmaceutical or cosmetic compositions preferably in the form of microcapsules or microparticles which include at least one active ingredient and an oil or a mixture of oils wherein the oil or mixture of oils alone or in combination with the active ingredient have a specific gravity higher than the specific gravity of water. The invention further relates to a process for preparing said compositions.

### FIELD OF THE INVENTION

The present invention relates to pharmaceutical or cosmetic compositions containing oils having a specific gravity higher than water. More particularly, the invention relates to compositions preferably in the form of microcapsules or microparticles which include at least one active ingredient and an oil or a mixture of oils wherein the oil or mixture of oils alone or in combination with the active ingredient have a specific gravity higher than the specific gravity of water. The invention further relates to a process for preparing said compositions.

### BACKGROUND OF THE INVENTION

It is often desirable to construct micro-domains within a pharmaceutical or cosmetic composition with the aim of separating incompatible ingredients, stabilizing sensitive components, gaining control over release profile, or preventing contact with the area of application. Many approaches have been developed for obtaining micro-domains, for example utilizing the formation of separate phases within the formulation, use of micelles, vesicles, liposomes, particles of organic nature, particles of inorganic nature and others. (See for example: Novel cosmetic Delivery Systems, S. Magdassi, E. Touitou Eds., Marcel Dekker Inc. 1999, M. N. V. Ravi Kumar, J. Pharm. Pharmaceutic. Sci. 3(2) 234-258, 2000 and Microspheres, Microcapsules & Liposomes, Vol. 2: Medical & Biotechnology Applications, R. Ashady, Ed. Citus Books, 1999)

U.S. Pat. No. 6,303,149 discloses a method for the preparation of sol-gel microcapsules containing organic compounds for various uses. This method was used to form silica microcapsules, capable of enhancing the stability of sensitive active ingredients while delivering them following their topical application, as revealed in WO 01/80823.

Notably, the core-shell structured sol-gel capsules facilitate high loading of the encapsulated matter (up to 80 wt. % at capsule size of about 1 micron). Having a diameter of about 1 micron, these microcapsules are transparent on the skin, and are pleasant to touch with no gritty feeling. It should be realized that a major issue in the development and use of such small particles is developing effective and economic methods and means to isolate and purify the obtained microcapsules.

The capsules formed according to U.S. Pat. No. 6,303,149 enable the encapsulation of poorly soluble agents such as the anti-acne drug benzoyl peroxide (BPO) in a suitable oil, making this active drug available upon application in a soluble form. This is in contrast to most formulations of this drug currently in use, where the BPO is delivered as solid crystals and its activity relies on dissolution processes after application on the skin. The encapsulation of BPO in the dissolved form is expected to afford less irritant and more effective treatment for acne than formulations that consist of BPO crystals.

U.S. Pat. No. 5,086,075 supports this approach, teaching the use of an auxiliary solvent for a BPO composition, which has a boiling point substantially greater than 100° C. Once the water evaporates following application this solvent assists in dissolution of BPO crystals, making this formulation more effective and less irritant than formulations that do not have the auxiliary solvent. This patent lists the requirements from the auxiliary solvent, to have sufficient solubility of BPO in it; to be miscible with water; to be non-reactive with BPO at room temperature; to evaporate slowly; and to be non-toxic and not dermatological irritating.

The requirements from a solvent for BPO encapsulation according to WO 01/80823 are somewhat different: it has to have sufficient solubility of BPO; it has to be non-reactive with BPO at room temperature; it has to be non-miscible with water to allow formation of oil in water emulsion; it has to be non-toxic and not dermatologically irritating; and it is greatly advantageous if this solvent is dense enough to allow separation of the formed capsules by precipitation or by industrial centrifuges as part of the synthetic procedure.

U.S. Pat. No. 6,238,650 discloses the encapsulation of sunscreen active ingredients in sol-gel microcapsules. The active ingredients are highly retained within the silica capsules, minimizing exposure of the skin to the active ingredient. Additional advantage of the encapsulation is the retaining of ingredients which are solid in nature, i.e. butylmethoxy dibenzoylmethane (BMDBM), benzophenones and benzylidene camphors, as these ingredients are difficult to formulate and tend to precipitate over time in many formulations. Containing these actives within the capsule preferably in a dissolved form ensures that no precipitation will occur in the formulation over time. Numerous patents suggest solvents for UV absorbers trying to address this issue. A special case is BMDBM, which is difficult to dissolve and is photochemically unstable. Consequently, many patents teach the use of solvents that enhance its photostability. Examples include U.S. Pat. No. 5,670,140, U.S. Pat. No. 5,788,954, U.S. Pat. No. 5,783,173, EP 0 848 944, U.S. Pat. No. 5,849,273, U.S. Pat. No. 6,350,894 that teach the use of branched chain hydroxy benzoates or branched chain benzoates for the purpose; U.S. Pat. No. 5,576,354, EP 0 514 491 and U.S. Pat. No. 5,587,150 teach the use of alkyl β-β-diphenylacrylates or α cyano-β-β-diphenylacrylates (including Octocrylene); FR 94 14930, EP 0 717 982 and U.S. Pat. No. 5,672,337 teach the use of amides as solvents and photostabilizers of BMDBM.

Nonetheless, all these patents are interested in formulating BMDBM in emulsions. None of these patents deal with encapsulation of this UV absorber. Naturally, none of them teach the requirements of a suitable solvent for this purpose.

EP 0 934 773 and U.S. Pat. No. 6,337,089 teach microcapsules containing core material and a capsule wall made of organopolysiloxane, and their production. In all examples discussed by this patent, the core consists of octyl methoxycinnamate (OMC) or OMC and BMDBM, where the OMC also serves as the solvent for BMDBM. OMC is indeed a good solvent for BMDBM, but the combination of these two UV absorbers is notorious for being photochemically unstable, as these actives degrade each other under UV radiation. EP 0 934 773 and U.S. Pat. No. 6,337,089 teach that a good method for isolating the formed capsules is by a centrifuge. However, centrifugation is only applicable if the density of the capsules differs sufficiently from that of the synthesis solution. Hence, solvents that solubilize BMDBM effectively, while being cosmetically acceptable and having density that will allow precipitation of the formed capsules (i.e. higher than that of the aqueous solution) support the production of BMDBM without OMC. BMDBM alone or with added photostabilizer is more stable than BMDBM dissolved in OMC. Furthermore, separate encapsulation of these incompatible ingredients is an effective way to obtain improved photostability, as disclosed in U.S. Pat. No. 6,436,375.

EP 0 941 761 and U.S. Pat. No. 6,251,313 also teach the preparation of microcapsules having shell walls of organopolysiloxane. According to the specifications of this patent, the microcapsules have a size of 0.5 to 1000 μm. In this case too, microcapsules of the lower size range specified, namely 0.5 to 5 μm are very difficult to separate using simple filtration methods. Therefore, achieving separation by precipitation or sedimentation is essential to make the production of such particles at industrial scale. FR 2 774 906 teaches encapsulation of an organic core by emulsion-precipitation of carbonated aluminate and aluminium chloride in the presence of surfactants to form aluminum hydroxycarbonate shell. This shell is further coated with metal oxide or metal hydroxide when desired. This patent, however, does not teach the importance of selecting a suitable oil to be included in the microcapsule's core and the control of the core density to facilitate effective separation of the formed microcapsules.

Therefore, there is still a need in the art for a simpler and lower in cost process, in which the microcapsules or microparticles can be easily isolated through precipitation or sedimentation in a centrifuge.

Additionally, there is a need to provide a process in which the active ingredient solubility remains constant and does not crystallize during the microcapsule's or microparticle's preparation and yet which enables high loading of the active ingredient.

It is of great advantage in the process of producing microcapsules or microparticles if a suitable solvent in which the solubility of the active agent is not changed with temperature alteration is selected, thus enabling temperature changes as part of the process for production of a stable product.

Various compositions such as hand and body lotions have been prepared using dipropelene glycol dibenzoate (For example, FinsolvR PG-22, available commercially from Fintex Inc.).

FinsolvR PG-22 is an efficient skin conditioning agent functioning as both a humectant and emollient, acting on the upper layers of the skin to enhance moisturization. In addition, due to the benzoate portion of the molecule, FinsolvR PG-22 is used as a solvent and solubilizer, aiding in the formulation of clear products. Due to its unique tactile properties, solubility in water/alcohol mixtures and refractive index, FINSOLV PG-22 is an excellent candidate for use in clear, personal care products such as antiperspirants and deodorants. In addition, FINSOLV PG-22 is an excellent additive in both leave-in and rinse off hair care formulations adding humectancy and moisture to the hair as well as considerable shine [Fintex technical data sheets].

None of the prior art references disclose or suggest the use of dipropelene glycol dibenzoate in compositions in which there is an advantage in selecting an oil or mixtures of oils having a specific gravity higher than the specific gravity of water, or in a mixture including an oil or mixture of oils in combination with at least one active agent wherein said mixture is characterized by having a specific gravity higher than the specific gravity of water. The use of dipropelene glycol dibenzoate as a component in the microcapsules' core or in microparticles is highly advantageous.

### DEFINITIONS

For purposes of this specification and the accompanying claims, the term “water” refers to pure water or to the water phase of the microcapsules' or microparticles' preparation. The water phase may contain additional solutes or solvents that may influence the water specific gravity.

For purposes of this specification and the accompanying claims, the term “peroxide” refers to an organic compound containing an oxygen-oxygen bond capable of cleaving and forming oxygen free-radicals.

For purposes of this specification and the accompanying claims, the term “microcapsules” refers to a core material including at least one active ingredient and an oil or a mixture of oils, wherein the core is coated by a coating shell.

For purposes of this specification and the accompanying claims, the term “sol-gel microcapsules” refers to a core material which is coated by a sol-gel coating shell.

For purposes of this specification and the accompanying claims, the term “microparticles” refers to a matrix system in which the active ingredient is embedded in the matrix carrier.

For purposes of this specification and the accompanying claims, the term “dipropelene glycol dibenzoate” refers to the oil of dipropelene glycol dibenzoate as defined in the CTFA International Cosmetic Dictionary and Handbook, 2000, available commercially from Fintex Inc. (Finsolv PG-22), Velsicol (Benzoflex 9-88 and Benzoflex 9-88 SG), Alzo (Dermol DPG-2B) and Pentagon (DPGDB) or from any other source.

For purposes of this specification and the accompanying claims, the term “Finsolv TPP” refers to the commercial product of Fintex Inc., which is a blend of three benzoate esters: C12-15 alkyl benzoate, dipropylene glycol dibenzoate and PPG-15 stearyl ether benzoate.

For purposes of this specification and the accompanying claims, the term “Finsolv TN” refers to C12-15 alkyl benzoate.

For purposes of this specification and the accompanying claims, the term “BMDBM” refers to butyl methoxydibenzoylmethane.

For purposes of this specification and the accompanying claims, the term “Finsolv BCR-111” refers to cetyl ricinoleate benzoate.

For purposes of this specification and the accompanying claims, the term “Abil EM 90” refers to Cetyl dimethicone copolyol.

For purposes of this specification and the accompanying claims, the term “Arlamol HD” refers to Heptamethylnonane.

For purposes of this specification and the accompanying claims, the term “Cetiol SN” refers to Cetearyl isononanoate.

For purposes of this specification and the accompanying claims, the terms “UV Pearls OMC” and “UV Pearls BMDBM” refer to sol-gel encapsulated octyl methoxycinnamate and sol-gel encapsulated BMDBM, as produced by Sol-Gel Technologies and available commercially from Merck KGaA.

For purposes of this specification and the accompanying claims, the term “Euxyl K-100” refers to a preservative agent which is a mixture of Benzyl Alcohol, Methylchloroisothiazolinone and Methyl isothiazolinone, available commercially from Scheulke & Mayr.

For purposes of this specification and the accompanying claims, the term “EDTA” refers to a chelating agent which is ethylenediamine tetraacetic acid.

For purposes of this specification and the accompanying claims, the term “Montane 80” refers to sorbitan monooleate sold under this trade name by Seppic. Equivalent products are sold under different names by other producers.

For purposes of this specification and the accompanying claims, the term “Tween 80” refers to polysorbate 80. Tween 80 is the trade name of this agent made by Uniqeuma Americas, however equivalent material is available from various suppliers under different trade names.

For purposes of this specification and the accompanying claims, the term “Schercemol GMIS” refers to glycerolisostearate.

For purposes of this specification and the accompanying claims, the chemical group “COO” refers to

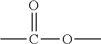

and “OOC” refers to

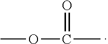

### SUMMARY OF THE INVENTION

According to one aspect of the present invention there is provided a pharmaceutical or cosmetic composition comprising at least one active ingredient in combination with an oil or a mixture of oils, wherein the oil or mixture of oils alone or in combination with the active ingredient have a specific gravity higher than the specific gravity of water.

According to further features in preferred embodiments of the invention described below, the active ingredient is dissolved in the oil or mixtures of oils at a concentration of about 0.1 to about 50 wt. %.

According to still further features in the described preferred embodiments, the active ingredient is dissolved in the oil or mixtures of oils at a concentration of about 2 to about 40 wt. %.

According to still further features in the described preferred embodiments, the oil or at least one oil in the oil mixtures is of the structural formula [I]:

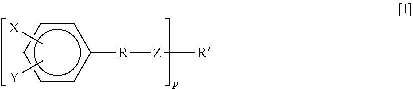

Wherein
R represents (CH₂)ₘ, where m is an integer from 0 to 3,
Z is selected from the group consisting of COO and OOC.
p is an integer from 1 to 3,
R′ is selected from the group consisting of an alkyl, aryl, arylalkyl, alkenyl, alcohol, polyol, benzoyl, benzoyl derivatives and azacyclic esters,
and wherein X and Y are independently selected from the group consisting of hydrogen, alkyl, alkoxy, amine, acetoxy ester, ether, halide, hydroxy, ketone, methylamine, and a nitro group.

According to still further features in the described preferred embodiments, the R′ includes 1 to 50 carbon atoms.

According to still further features in the described preferred embodiments, the X and Y independently include 0 to 10 carbon atoms.

According to still further features in the described preferred embodiments, the oil of formula [I] as defined by X, Y, m, Z, R′ and p is selected from the group consisting of:

X, Y=H, m=0, Z=COO, R′=(CH₂)₃O(CH₂)₃, p=2;
X, Y=H, m=0, Z=COO, R′=(CH₂)₂O(CHO₂, p=2;
X, Y=H, m=0, Z=COO, R′=CH₃, p=1;
X, Y=H, m=0, Z=COO, R′=CH₂C₆H₆, p=1;
X, Y=H, m=0, Z=COO, R′=CH₂CH₃, p=1;
X, Y=H, m=0, Z=COO, R′=C₆H₅, p=1;
X, Y=H, m=1, Z=COO, R′=C₆H₅CH₂CH₃, p=1;
X=2-OH, Y=H, m=0, Z=COO, R′=C₆H₅, p=1;
X=2-OH, Y=H, m=0, Z=COO, R′=CH₃, p=1;
X, Y=H, m=0, Z=COO, R′=(CH₂)₃, p=3;
X, Y=H, m=0, Z=COO, R′=CH(CH₃)₂, p=1;
X, Y=H, m=0, Z=COO, R′=(CH₂)₂CH₃, p=1;
X=2-OH, Y=H, m=0, Z=COO, R′=CH₂H₅, p=1;
X=4-OCH₃, Y=H, m=0, Z=COO, R′=CH₂CH₃, p=1;
X, Y=H, m=1, Z=OOC, R′=CH₂CH₃, p=1;
X=CH₃O, Y=H, m=1, Z=OOC, R′=CH₃, p=1;
X=CH₃CH₂, Y=H, m=0, Z=OOC, R′=CH₃, p=1;
and mixtures thereof.

According to still further features in the described preferred embodiments, the mixture of oils comprises
(a) at least one oil having a specific gravity lower than the specific gravity of water;
(b) at least one oil having a specific gravity higher than the specific gravity of water;
wherein the mixture of oils alone or in combination with the active ingredient have a specific gravity higher than the specific gravity of water.

According to still further features in the described preferred embodiments, the mixture of oils comprises
(a) at least one oil selected from the group consisting of C12-C15 alkyl benzoate, isostraryl benzoate, PPG-15 stearyl ether benzoate, octyldodecyl benzoate, stearyl benzoate, methyl gluceth-20 benzoate, poloxamer 182 dibenzoate, poloxamer 105 benzoate, transcutol, dioctyl malate, diethylhexylmaleate, diethylhexylsebacate, diethylhexyladipate, diisopropyladipate, diisopropylsebacate, diisopropylmaleate, ethylhexylsalicylate, tridecylsalicylate, butyloctylsalycilate, isopropylmyristate and mixture thereof;
(b) at least one oil as defined in the present invention by structural formula [I];
wherein the mixture of oils alone or in combination with the active ingredient have a specific gravity higher than the specific gravity of water.

According to still further features in the described preferred embodiments, the oil is a mixture of dipropylene glycol dibenzoate, ethyl salicylate and ethylbenzoate.

According to still further features in the described preferred embodiments, the active ingredient is selected from the group consisting of a sunscreen agent, a dermatological agent, a pharmaceutical agent, a vitamin, an anti-inflammatory agent, an analgesic, an anti-fungal agent, an anti-biotic, an anti-viral agent, an anti-acne agent, an anti histamine, an enzyme, a co-enzyme, a humectant, a dermatological agent, an insect repellent, a perfume, a color, a dye, a skin whitening agent, an aromatic oil, a flavoring agent, a dental agent, an anti-parasitic agent, a muscle relaxant, a steroid, a hormone, an astringent, a toner, an anti-oxidant, and mixtures thereof.

According to still further features in the described preferred embodiments, the active ingredient is a sunscreen agent.

According to still further features in the described preferred embodiments, the sunscreen agent is selected from the group consisting of a UVA absorber, a UVB absorber or a combination thereof.

According to still further features in the described preferred embodiments, the sunscreen agent is selected from the group consisting of benzophenones, 4,4′-methoxy-t-butyldibenzoylmethane, 4-isopropyl dibenzoylmethane, 4-methylbenzylidene camphor, 3-benzylidene camphor, polyacrylamidomethyl benzylidene camphor and mixtures thereof.

According to still further features in the described preferred embodiments, the benzophenones are selected from the group consisting of benzophenone-3, benzophenone-1, benzophenone-2, benzophenone-6 and benzophenone-8 and mixtures thereof.

According to still further features in the described preferred embodiments, the dermatological agent is a peroxide.

According to still further features in the described preferred embodiments, the peroxide is selected from the group consisting of benzoyl peroxide, urea peroxide and mixtures thereof.

According to still further features in the described preferred embodiments, the composition is selected from the group consisting of an ointment, a cream, a lotion, a microcapsules' dispersion, a microparticles' dispersion, an oil, a gel, a solid stick, a milk, an aerosol, a spray, a powder, a foam, a mousse, a shampoo, a hair conditioner, a lacquer, a make-up, a soap, a paste, a lipstick, a lipcare product, an eyeshadow, a blusher, a presun or aftersun preparations, a hair colorant, a hair highlighter preparation, an astringent and a cleanser.

According to another aspect of the present invention there is provided a microcapsular composition comprising:
(a) a core, including at least one active ingredient in combination with an oil or mixture of oils, wherein the oil or mixture of oils alone or in combination with the active ingredient have a specific gravity higher than the specific gravity of water; and
(b) at least one microcapsular shell encapsulating the core.

According to further features in preferred embodiments of the invention described below, the oil or at least one oil in the oil mixtures is of the structural formula [I]:

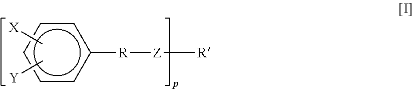

Wherein
R represents (CH₂)ₘ, where m is an integer from 0 to 3,
Z is selected from the group consisting of COO and OOC.
p is an integer from 1 to 3,
R′ is selected from the group consisting of an alkyl, aryl, arylalkyl, alkenyl, alcohol, polyol, benzoyl, benzoyl derivatives and azacyclic esters,
and wherein X and Y are independently selected from the group consisting of hydrogen, alkyl, alkoxy, amine, acetoxy ester, ether, halide, hydroxy, ketone, methylamine, and a nitro group.

According to still further features in the described preferred embodiments, the R′ includes 1 to 50 carbon atoms.

According to still further features in the described preferred embodiments, the X and Y independently include 0 to 10 carbon atoms.

According to still further features in the described preferred embodiments, the oil of formula [I] as defined by X, Y, m, Z, R′ and p is selected from the group consisting of:

X, Y=H, m=0, Z=COO, R′=(CH₂)₃O(CH₂)₃, p=2;
X, Y=H, m=0, Z=COO, R′=(CH₂)₂O(CH₂)₂, p=2;
X, Y=H, m=0, Z=COO, R′=CH₃, p=1;
X, Y=H, m=0, Z=COO, R′=CH₂C₆H₆, p=1;
X, Y=H, m=0, Z=COO, R′=CH₂CH₃, p=1;
X, Y=H, m=0, Z=COO, R′=C₆H₅, p=1;
X, Y=H, m=1, Z=COO, R′=C₆H₅CH₂CH₃, p=1;
X=2-OH, Y=H, m=0, Z=COO, R′=C₆H₅, p=1;
X=2-OH, Y=H, m=0, Z=COO, R′=CH₃, p=1;
X, Y=H, m=0, Z=COO, =(CH₂)₃, p=3;
X, Y=H, m=0, Z=COO, R′ CH(CH₃)₂, p=1;
X, Y=H, m=0, Z=COO, R′=(CH₂)₂CH₃, p=1;
X=2-OH, Y=H, m=0, Z=COO, R′=CH₂H₅, p=1;
X=4-OCH₃, Y=H, m=0, Z=COO, R′=CH₂CH₃, p=1;
X, Y=H, m=1, Z=OOC, R′=CH₂CH₃, p=1;
X=CH₃O, Y=H, m=1, Z=OOC, R′=CH₃, p=1;
X=CH₃CH₂, Y=H, m=0, Z=OOC, R′=CH₃, p=1;
and mixtures thereof.

According to still further features in the described preferred embodiments, the mixture of oils comprises
(a) at least one oil having a specific gravity lower than the specific gravity of water;
(b) at least one oil having a specific gravity higher than the specific gravity of water;
wherein the mixture of oils alone or in combination with the active ingredient have a specific gravity higher than the specific gravity of water.

According to still further features in the described preferred embodiments, the mixture of oils comprises
(a) at least one oil selected from the group consisting of C12-C15 alkyl benzoate, isostraryl benzoate, PPG-15 stearyl ether benzoate, octyldodecyl benzoate, stearyl benzoate, methyl gluceth-20 benzoate, poloxamer 182 dibenzoate, poloxamer 105 benzoate, transcutol, dioctyl malate, diethylhexylmaleate, diethylhexylsebacate, diethylhexyladipate, diisopropyladipate, diisopropylsebacate, diisopropylmaleate, ethylhexylsalicylate, tridecylsalicylate, butyloctylsalycilate, isopropylmyristate and mixture thereof;
(b) at least one oil as defined in the present invention by structural formula [I];
   wherein the mixture of oils alone or in combination with the active ingredient have a specific gravity higher than the specific gravity of water.

According to still further features in the described preferred embodiments, the oil is a mixture of dipropylene glycol dibenzoate, ethyl salicylate and ethylbenzoate.

According to still further features in the described preferred embodiments, the composition is in the form of sol-gel microcapsules.

According to still further features in the described preferred embodiments, the active ingredient is dissolved in the oil or mixtures of oils at a concentration of about 0.1 to about 50 wt. %.

According to still further features in the described preferred embodiments, the active ingredient is dissolved in the oil or mixtures of oils at a concentration of about 2 to about 40 wt. %.

According to still further features in the described preferred embodiments, the active ingredient is selected from the group consisting of a suncscreen agent, a dermatological agent, a pharmaceutical agent, a vitamin, an anti-inflammatory agent, an analgesic, an anti-fungal agent, an anti-biotic, an anti-viral agent, an anti-acne agent, an anti histamine, an enzyme, a co-enzyme, a humectant, a dermatological agent, an insect repellent, a perfume, a color, a dye, a skin whitening agent, an aromatic oil, a flavoring agent, a dental agent, an anti-parasitic agent, a muscle relaxant, a steroid, a hormone, an astringent, a toner, an anti-oxidant, and mixtures thereof.

According to still further features in the described preferred embodiments, the active ingredient is a sunscreen agent.

According to still further features in the described preferred embodiments, the sunscreen agent is selected from the group consisting of a UVA absorber, a UVB absorber or a combination thereof.

According to still further features in the described preferred embodiments, the sunscreen agent is selected from the group consisting of benzophenones, 4,4′-methoxy-t-butyldibenzoylmethane, 4-isopropyl dibenzoylmethane, 4-methylbenzylidene camphor, 3-benzylidene camphor, polyacrylamidomethyl benzylidene camphor and mixtures thereof.

According to still further features in the described preferred embodiments, the benzophenones are selected from the group consisting of benzophenone-3, benzophenone-1, benzophenone-2, benzophenone-6 and benzophenone-8 and mixtures thereof.

According to still further features in the described preferred embodiments, the dermatological agent is a peroxide.

According to still further features in the described preferred embodiments, the peroxide is selected from the group consisting of benzoyl peroxide, urea peroxide and mixtures thereof.

According to still further features in the described preferred embodiments, the microcapsules are dispersed in a pharmaceutical or a cosmetic composition.

According to yet another aspect of the present invention there is provided a microparticulate highly porous matrix composition comprising a matrix carrier and at least one active ingredient dissolved in an oil or mixture of oils, wherein the oil or mixture of oils alone or in combination with the active ingredient have a specific gravity higher than the specific gravity of water.

According to further features in preferred embodiments of the invention described below, the oil or at least one oil in the oil mixtures is of the structural formula [I]:

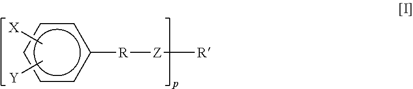

Wherein
R represents (CH₂)ₘ, where m is an integer from 0 to 3,
Z is selected from the group consisting of COO and OOC.
p is an integer from 1 to 3,
R′ is selected from the group consisting of an alkyl, aryl, arylalkyl, alkenyl, alcohol, polyol, benzoyl, benzoyl derivatives and azacyclic esters,
and wherein X and Y are independently selected from the group consisting of hydrogen, alkyl, alkoxy, amine, acetoxy ester, ether, halide, hydroxy, ketone, methylamine, and a nitro group.

According to still further features in the described preferred embodiments, the R′ includes 1 to 50 carbon atoms.

According to still further features in the described preferred embodiments, the X and Y independently include 0 to 10 carbon atoms.

According to still further features in the described preferred embodiments, the oil of formula [I] as defined by X, Y, m, Z, R′ and p is selected from the group consisting of:

X, Y=H, m=0, Z=COO, R′=(CH₂)₃O(CH₂)₃, p=2;
X, Y=H, m=0, Z=COO, R′=(CH₂)₂O(CH₂)₂, p=2;
X, Y=H, m=0, Z=COO, R′=CH₃, p=1;
X, Y=H, m=0, Z=COO, R′=CH₂C₆H₆, p=1;
X, Y=H, m=0, Z=COO, R′=CH₂CH₃, p=1;
X, Y=H, m=0, Z=COO, R′=C₆H₅, p=1;
X, Y=H, m=1, Z=COO, R′=C₆H₅CH₂CH₃, p=1;
X=2-OH, Y=H, m=0, Z=COO, R′=C₆H₅, p=1,
X=2-OH, Y=H, m=0, Z=COO, R′=CH₃, p=1;
X, Y=H, m=0, Z=COO, R′=(CH₂)₃, p=3;
X, Y=H, m=0, Z=COO, R′=CH(CH₃)₂, p=1;
X, Y=H, m=0, Z=COO, R′=(CH₂)₂CH₃, p=1;
X=2-OH, Y=H, m=0, Z=COO, R′=CH₂H₅, p=1;
X=4-OCH₃, Y=H, m=0, Z=COO, R′=CH₂CH₃, p=1;
X, Y=H, m=1, Z=OOC, R′=CH₂CH₃, p=1;
X=CH₃O, Y=H, m=1, Z=OOC, R′=CH₃, p=1;
X=CH₃CH₂, Y=H, m=0, Z=OOC, R′=CH₃, p=1;
and mixtures thereof.

According to still further features in the described preferred embodiments, the mixture of oils comprises
(a) at least one oil having a specific gravity lower than the specific gravity of water;
(b) at least one oil having a specific gravity higher than the specific gravity of water;
wherein the mixture of oils alone or in combination with the active ingredient have a specific gravity higher than the specific gravity of water.

According to still further features in the described preferred embodiments, the mixture of oils comprises
(a) at least one oil selected from the group consisting of C12-C15 alkyl benzoate, isostraryl benzoate, PPG-15 stearyl ether benzoate, octyldodecyl benzoate, stearyl benzoate, methyl gluceth-20 benzoate, poloxamer 182 dibenzoate, poloxamer 105 benzoate, transcutol, dioctyl malate, diethylhexylmaleate, diethylhexylsebacate, diethylhexyladipate, diisopropyladipate, diisopropylsebacate, diisopropylmaleate, ethylhexylsalicylate, tridecylsalicylate, butyloctylsalycilate, isopropylmyristate and mixture thereof;
(b) at least one oil as defined in the present invention by structural formula [I];
   wherein the mixture of oils alone or in combination with the active ingredient have a specific gravity higher than the specific gravity of water.

According to still further features in the described preferred embodiments, the oil is a mixture of dipropylene glycol dibenzoate, ethyl salicylate and ethylbenzoate.

According to still further features in the described preferred embodiments, the active ingredient is dissolved in the oil or mixtures of oils at a concentration of about 0.1 to about 50 wt. %.

According to still further features in the described preferred embodiments, the active ingredient is dissolved in the oil or mixtures of oils at a concentration of about 2 to about 40 wt. %.

According to still further features in the described preferred embodiments, the active ingredient is selected from the group consisting of a suncscreen agent, a dermatological agent, a pharmaceutical agent, a vitamin, an anti-inflammatory agent, an analgesic, an anti-fungal agent, an anti-biotic, an anti-viral agent, an anti-acne agent, an anti histamine, an enzyme, a co-enzyme, a humectant, a dermatological agent, an insect repellent, a perfume, a color, a dye, a skin whitening agent, an aromatic oil, a flavoring agent, a dental agent, an anti-parasitic agent, a muscle relaxant, a steroid, a hormone, an astringent, a toner, an anti-oxidant, and mixtures thereof.

According to still further features in the described preferred embodiments, the active ingredient is a sunscreen agent.

According to still further features in the described preferred embodiments, the sunscreen agent is selected from the group consisting of a UVA absorber, a UVB absorber or a combination thereof.

According to still further features in the described preferred embodiments, the sunscreen agent is selected from the group consisting of benzophenones, 4,4′-methoxy-t-butyldibenzoylmethane, 4-isopropyl dibenzoylmethane, 4-methylbenzylidene camphor, 3-benzylidene camphor, polyacrylamidomethyl benzylidene camphor and mixtures thereof.

According to still further features in the described preferred embodiments, the benzophenones are selected from the group consisting of benzophenone-3, benzophenone-1, benzophenone-2, benzophenone-6 and benzophenone-8.

According to still further features in the described preferred embodiments, the dermatological agent is a peroxide.

According to still further features in the described preferred embodiments, the peroxide is selected from the group consisting of benzoyl peroxide, urea peroxide and mixtures thereof.

According to still further features in the described preferred embodiments, the microparticles are dispersed in a pharmaceutical or a cosmetic composition.

According to still another aspect of the present invention there is provided a process for preparing a pharmaceutical or cosmetic composition as described in the present invention comprising at least the step of dissolving an active ingredient in an oil or mixture of oils, wherein the oil or mixture of oils alone or in combination with the active ingredient have a specific gravity higher than the specific gravity of water.

According to an additional aspect of the present invention there is provided a process for preparing a microcapsular composition comprising the steps of:
(a) dissolving an active ingredient in a mixture including:
   (i) an oil or mixture of oils, wherein the oil or mixture of oils alone or in combination with the active ingredient have a specific gravity higher than the specific gravity of water; and
   (ii) sol-gel precursors to form a hydrophobic solution;
(b) emulsifying the hydrophobic solution in an aqueous solution under high shear forces;
(c) mixing and stirring the emulsion obtained in step (b) with a second aqueous solution at a predetermined pH to obtain the microcapsules; and
(d) isolating the obtained microcapsules through precipitation or sedimentation in a centrifuge.

According to yet additional aspect of the present invention there is provided a method of treating a medical condition in a human patient, the medical condition is selected from the group consisting of a skin, hair, ear, mucosal membrane, rectal, nasal or dental disease, disorder or condition, the method comprising the step of administering a composition as described in the present invention to a patient in need of such treatment.

According to further features in preferred embodiments of the invention described below, the condition is selected from the group consisting of acne, psoriasis, seborrea, bacterial infection, viral infection, fungal infection, inflammation, aging signs, dandruff and cavity.

### DETAILED DESCRIPTION OF THE INVENTION

The present invention provides a pharmaceutical or cosmetic composition comprising at least one active ingredient in combination with an oil or a mixture of oils wherein the oil or mixture of oils alone or in combination with the active ingredient have a specific gravity higher than the specific gravity of water.

According to the present invention, the term “water” refers to pure water or to the water phase of the microcapsules' or microparticulate process preparation. The water phase may contain additional solvents or solutes that may influence the water specific gravity. The solute concentration maybe up to about 10% by weight of the water weight. For example 10% by wt. of ethanol reduces the water specific gravity to 0.9819.10% by wt. of ammonia reduces the water specific gravity to 0.9575.10% by wt. of ammonium chloride increases the water specific gravity to 1.0286.10% by wt. of sodium acetate increases the water specific gravity to 1.0495.10% by wt. of calcium chloride increases the water specific gravity to 1.0835. The influence of various solvents and solutes on the specific gravity of water are described in CRC Handbook of Chemistry and Physics, 66^{th} edition (1985-1986).

Preferably the water specific gravity in the present invention is from about 0.95 to about 1.10. More preferably, the water specific gravity in the present invention is from about 0.97 to about 1.08.

The active ingredient is preferably present in the oil or mixture of oils in the dissolved state. The active ingredient may also be present in the dissolved and dispersed state. However, in order to provide therapeutic concentrations of the active ingredient in the site of application it is highly advantageous to have compositions wherein the active ingredient is dissolved in the composition.

The active ingredient may be dissolved in the oil or mixtures of oils at a concentration of about 0.1 to about 50 wt. %, preferably at a concentration of about 2 to about 40%, more preferably at a concentration of about 5 to about 35%.

Surprisingly, it was found that the solubility of the active agents are not significantly changed with temperature in a specific combination of solvents including dipropylene glycol dibenzoate/Ethyl Salicylate/Ethylbenzoate, used in the present invention. This is highly advantageous since it enables the production of a stable product which is not affected by temperature changes during its production. The active ingredient maintains its solubility during processing.

The oils of the pharmaceutical or cosmetic compositions of the present invention, characterized by having a specific gravity higher than the specific gravity of water, were found to be highly advantageous because of their good solubility properties.

The oil or at least one oil in the oil mixtures is of the structural formula [I]:

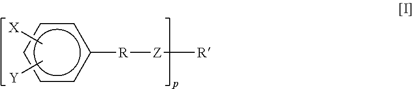

Wherein
R represents (CH₂)ₘ, where m is an integer from 0 to 3,
Z is selected from the group consisting of COO and OOC.
p is an integer from 1 to 3,
R′ is selected from the group consisting of an alkyl, aryl, arylalkyl, alkenyl, alcohol, polyol, benzoyl, benzoyl derivatives and azacyclic esters,
and wherein X and Y are independently selected from the group consisting of hydrogen, alkyl, alkoxy, amine, acetoxy ester, ether, halide, hydroxy, ketone, methylamine, and a nitro group.
   Preferably R′ includes 1 to 50 carbon atoms.
   Preferably X and Y independently include 0 to 10 carbon atoms.

The oil may be for example, dipropylene glycol dibenzoate (X, Y=H, m=0, R′=(CH₂)₃O(CH₂)₃, p=2), diethylene glycol dibenzoate (X, Y=H, m=0, Z=COO, R′=(CH₂)₂O(CH₂)₂, p=2), methyl benzoate (X, Y=H, m=0, Z=COO, R′=CH₃, p=1), benzyl benzoate (X, Y=H, m=0, Z=COO, R′=CH₂C₆H₆, p=1), ethyl benzoate (X, Y=H, m=0, Z=COO, R′=CH₂CH₃, p=1), phenyl benzoate (X, Y=H, m=0, Z=COO, R′=C₆H₅, p=1), phenyl ethyl benzoate (X, Y=H, m=1, Z=COO, R′=C₆H₅CH₂CH₃, p=1), phenyl salicylate (X=2-OH, Y=H, m=0, Z=COO, R′=C₆H₅, p=1), methyl salicylate (X=2-OH, Y=H, m=0, Z=COO, R′=CH₃, p=1), glycerol tribenzoate (X, Y=H, m=0, Z=COO, R′=(CH₂)₃, p=3), isopropyl benzoate (X, Y=H, m=0, Z=COO, R′=CH(CH₃)₂, p=1), propyl benzoate (X, Y=H, m=0, Z=COO, R′=(CH₂)₂CH₃, p=1), ethyl salicylate (X=2-OH, Y=H, m=0, Z=COO, R′=C₂H₅, p=1), Ethyl Anisate (X=4-OCH₃, Y=H, m=0, Z=COO, R′=CH₂CH₃, p=1), benzyl propionate (X, Y=H, m=1, Z=OOC, R′=CH₂CH₃, p=1), anisyl acetate (X=CH₃O, Y=H, m=1, Z=OOC, R′=CH₃, p=1), phenyl ethyl acetate (X=CH₃CH₂, Y=H, m=0, Z=OOC, R′=CH₃, p=1) or mixtures thereof.

In a preferred embodiment, the oil is dipropylene glycol dibenzoate or a mixture of dipropylene glycol dibenzoate with ethyl benzoate and ethyl salicylate.

When a mixture including dipropylene glycol dibenzoate, ethyl salicylate and ethyl benzoate is used, the weight fraction of dipropylene glycol dibenzoate is from about 31 to about 88.5; the weight fraction of ethyl salicylate is from about 7 to about 42; the weight fraction of ethyl benzoate is from about 4.5 to about 27.

Most preferably the weight fractions of dipropylene glycol dibenzoate: ethyl salicylate: ethyl benzoate are 77:14:9 respectively.

In another preferred embodiment the oil is methyl salicylate or ethyl salicylate.

In yet another preferred embodiment the oil is selected from the group consisting of ethyl benzoate, benzyl proprionate, anisyl acetate, phenyl ethyl benzoate, phenyl ethyl acetate, ethyl anisate and mixtures thereof.

The composition of the present invention may comprise a mixture of oils including:
(a) at least one oil having a specific gravity lower than the specific gravity of water;
(b) at least one oil having a specific gravity higher than the specific gravity of water;
   wherein the mixture of oils have a specific gravity higher than the specific gravity of water.

The compositions of the present invention may include for example a mixture of oils comprising:
(a) at least one oil selected from the group consisting of C12-C15 alkyl benzoate, isostraryl benzoate, PPG-15 stearyl ether benzoate, octyldodecyl benzoate, stearyl benzoate, methyl gluceth-20 benzoate, poloxamer 182 dibenzoate, poloxamer 105 benzoate, transcutol, dioctyl malate, diethylhexylmaleate, diethylhexylsebacate, diethylhexyladipate, diisopropyladipate, diisopropylsebacate, diisopropylmaleate, ethylhexylsalicylate, tridecylsalicylate, butyloctylsalycilate, isopropylmyristate and mixture thereof;
(b) at least one oil at as defined in the present invention by formula (I)
   wherein the mixture of oils alone or in combination with the active ingredient have a specific gravity higher than the specific gravity of water.

There may be cases in which the oil or mixture of oils used in the compositions of the present invention have a specific gravity lower than water, however in combination with the active ingredient the specific gravity increases and results in a combination having a specific gravity higher than the specific gravity of water.

The active ingredient may be for example a suncscreen agent, a dermatological agent, a pharmaceutical agent, a vitamin, an anti-inflammatory agent, an analgesic, an anti-fungal agent, an anti-biotic, an anti-viral agent, an anti-acne agent, an anti histamine, an enzyme, a co-enzyme, a humectant, a dermatological agent, an insect repellent, a perfume, a color, a dye, a skin whitening agent, an aromatic oil, a flavoring agent, a dental agent, an anti-parasitic agent, a muscle relaxant, a steroid, a hormone, an astringent, a toner, an anti-oxidant, or mixtures thereof.

In one preferred embodiment of the current invention the active is a sunscreen agent.

The sunscreen agent may be for example a UVA absorber, a UVB absorber or a combination thereof.

The sunscreen agent may be for example, benzophenones, 4,4′-methoxy-t-butyldibenzoylmethane, 4-isopropyl dibenzoylmethane, 4-methylbenzylidene camphor, 3-benzylidene camphor, Polyacrylamidomethyl benzylidene camphor.

The benzophenones are preferably oxybenzone (benzophenone-3), benzophenone-1, benzophenone-2, benzophenone-6 and benzophenone-8. Preferably the dermatological agent is a peroxide.

The peroxides include but not limited to peroxyacids of carboxylic acids, peroxyesters of carboxylic acids and the dimeric product of carboxylic peroxyacids. Exemplary peroxides include t-butyl peroxyesters of straight and branched chain aliphatic carboxylic acids, and dimeric peroxides such as lauroyl peroxide and benzoyl peroxide. In addition the peroxides include urea peroxide. A preferred peroxide for use in the present invention is benzoyl peroxide and urea peroxide.

In the present invention, dipropylene glycol dibenzoate was found to be a good solvent for benzoyl peroxide. This property of dipropylene glycol dibenzoate supports its use as a solubilizer for the pharmaceutical agent, to obtain a non-irritant benzoyl peroxde preparation for the treatment of Acne.

The compositions of the present invention may be in the form of an ointment, a cream, a lotion, a microcapsules' dispersion, a microparticles' dispersion, an oil, a gel, a solid stick, a milk, an aerosol, a spray, a powder, a foam, a mousse, a shampoo, a hair conditioner, a lacquer, a make-up, a soap, a paste, a lipstick, a lipcare product, an eyeshadow, a blusher, a presun or aftersun preparations, a hair colorant, a hair highlighter preparation, an astringent and a cleanser.

The present invention further provides a microcapsular composition comprising:
(a) a core, including at least one active ingredient and an oil or mixture of oils, wherein the oil or mixture of oils alone or in combination with the active ingredient have a specific gravity higher than the specific gravity of water; and
(b) at least one microcapsular shell encapsulating the core.
   The oil or at least one oil in the oil mixtures is of the structural formula [I]:

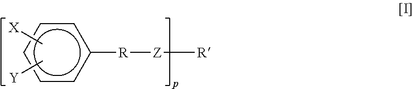

Wherein
R represents (CH₂)ₘ, where m is an integer from 0 to 3,
Z is selected from the group consisting of COO and OOC.
p is an integer from 1 to 3,
R′ is selected from the group consisting of an alkyl, aryl, arylalkyl, alkenyl, alcohol, polyol, benzoyl, benzoyl derivatives and azacyclic esters,
and wherein X and Y are independently selected from the group consisting of hydrogen, alkyl, alkoxy, amine, acetoxy ester, ether, halide, hydroxy, ketone, methylamine, and a nitro group.
   Preferably R′ includes 1 to 50 carbon atoms.
   Preferably X and Y independently include 0 to 10 carbon atoms.

The oil may be for example, dipropylene glycol dibenzoate (X, Y=H, m=0, Z=COO, R′=(CH₂)₃O(CH₂)₃, p=2), diethylene glycol dibenzoate (X, Y=H, m=0, Z=COO, R′=(CH₂)₂O(CH₂)₂, p=2), methyl benzoate (X, Y=H, m=0, Z=COO, R′=CH₃, p=1), benzyl benzoate (X, Y=H, m=0, Z=COO, R′=CH₂C₆H₆, p=1) ethyl benzoate (X, Y=H, m=0, Z=COO, R′=CH₂CH₃, p=1), phenyl benzoate (X, Y=H, m=0, Z=COO, R′=C₆H₅, p=1), phenyl ethyl benzoate (X, Y=H, m=1, Z=COO, R′=C₆H₅CH₂CH₃, p=1), phenyl salicylate (X=2-OH, Y=H, m=0, Z=COO, R′=C₆H₅, p=1), methyl salicylate (X=2-OH, Y=H, m=0, Z=COO, R′=CH₃, p=1), glycerol tribenzoate (X, Y=H, m=0, Z=COO, R′=(CH₂)₃, p=3), isopropyl benzoate (X, Y=H, m=0, Z=COO, R′=CH(CH₃)₂, p=1), propyl benzoate (X, Y=H, m=0, Z=COO, R′=(CH₂)₂CH₃, p=1), ethyl salicylate (X=2-OH, Y=H, m=0, Z=COO, R′=C₂H₅, p=1), Ethyl Anisate (X=4-OCH₃, Y=H, m=0, Z=COO, R′=CH₂CH₃, p=1), benzyl propionate (X, Y=H, m=1, Z=OOC, R′=CH₂CH₃, p=1), anisyl acetate (X=CH₃O, Y=H, m=1, Z=OOC, R′=CH₃, p=1), phenyl ethyl acetate (X=CH₃CH₂, Y=H, m=0, Z=OOC, R′=CH₃, p=1) or mixtures thereof.

In a preferred embodiment, the oil is dipropylene glycol dibenzoate or a mixture of dipropylene glycol dibenzoate with ethyl benzoate and ethyl salicylate.

When a mixture including dipropylene glycol dibenzoate, ethyl salicylate and ethyl benzoate is used, the weight fraction of dipropylene glycol dibenzoate is from about 31 to about 88.5; the weight fraction of ethyl salicylate is from about 7 to about 42; the weight fraction of ethyl benzoate is from about 4.5 to about 27.

Most preferably the weight fractions of dipropylene glycol dibenzoate: ethyl salicylate: ethyl benzoate are 77:14:9 respectively.

In another preferred embodiment the oil is methyl salicylate or ethyl salicylate.

In yet another preferred embodiment the oil is selected from the group consisting of ethyl benzoate, benzyl proprionate, anisyl acetate, phenyl ethyl benzoate, phenyl ethyl acetate, ethyl anisate and mixtures thereof.

The microcapsular composition of the present invention may comprise a mixture of oils including:
(a) at least one oil having a specific gravity lower than the specific gravity of water;
(b) at least one oil having a specific gravity higher than the specific gravity of water;
wherein the mixture of oils have a specific gravity higher than the specific gravity of water.

The microcapsular compositions of the present invention may include for example a mixture of oils comprising:
(a) at least one oil selected from the group consisting of C12-C15 alkyl benzoate, isostraryl benzoate, PPG-15 stearyl ether benzoate, octyldodecyl benzoate, stearyl benzoate, methyl gluceth-20 benzoate, poloxamer 182 dibenzoate, poloxamer 105 benzoate, transcutol, dioctyl malate, diethylhexylmaleate, diethylhexylsebacate, diethylhexyladipate, diisopropyladipate, diisopropylsebacate, diisopropylmaleate, ethylhexylsalicylate, tridecylsalicylate, butyloctylsalycilate, isopropylmyristate and mixture thereof;
(b) at least one oil at as defined in the present invention by formula (I)
   wherein the mixture of oils alone or in combination with the active ingredient have a specific gravity higher than the specific gravity of water.

The microcapsular composition is preferably in the form of sol-gel microcapsules.

In a preferred embodiment of this invention, the sol-gel microcapsules are silica or organically modified silica microcapsules, produced through the sol-gel process disclosed in U.S. Pat. No. 6,303,149 (U.S. application Ser. No. 09/372,176). The term “organically modified silica microcapsules” refers to sol-gel microcapsules which are obtained when the sol-gel precursors are of the formula M(R)ₙ(P)ₘ, wherein M is Si, R is a hydrolyzable substituent, n is an integer from 2 to 5, P is a non polymerizable substituent and m is and integer from 1 to 6.

The sol-gel microcapsules may also be produced through a sol-gel process wherein the sol-gel precursors are of the formula M(R)ₙ(P)ₘ, wherein M is Ti, Fe, Zn, Zr or Al, R is a hydrolyzable substituent, n is an integer from 2 to 6, P is a non polymerizable substituent and m is and integer from 0 to 6.

The active ingredient may be dissolved in the oil or mixtures of oils at a concentration of about 0.1 to about 50 wt. %, preferably at a concentration of about 2 to about 40%, more preferably at a concentration of about 5 to about 35%.

The active ingredient may be for example a suncscreen agent, a dermatological agent, a pharmaceutical agent, a vitamin, an anti-inflammatory agent, an analgesic, an anti-fungal agent, an anti-biotic, an anti-viral agent, an anti-acne agent, an anti histamine, an enzyme, a co-enzyme, a humectant, a dermatological agent, an insect repellent, a perfume, a color, a dye, a skin whitening agent, an aromatic oil, a flavoring agent, a dental agent, an anti-parasitic agent, a muscle relaxant, a steroid, a hormone, an astringent, a toner, an anti-oxidant, or mixtures thereof. In one preferred embodiment of the current invention the active is a sunscreen agent.

The sunscreen agent may be for example a UVA absorber, a UVB absorber or a combination thereof.

The sunscreen agent may be for example, benzophenones, 4,4′-methoxy-t-butyldibenzoylmethane, 4-isopropyl dibenzoylmethane, 4-methylbenzylidene camphor, 3-benzylidene camphor, Polyacrylamidomethyl benzylidene camphor.

The benzophenones are preferably oxybenzone (benzophenone-3), benzophenone-1, benzophenone-2, benzophenone-6 and benzophenone-8. Preferably the dermatological agent is a peroxide.

The peroxides include but not limited to peroxyacids of carboxylic acids, peroxyesters of carboxylic acids and the dimeric product of carboxylic peroxyacids. Exemplary peroxides include t-butyl peroxyesters of straight and branched chain aliphatic carboxylic acids, and dimeric peroxides such as lauroyl peroxide and benzoyl peroxide. In addition the peroxides include urea peroxide. A preferred peroxide for use in the present invention is benzoyl peroxide and urea peroxide.

The present invention further provides a microparticulate highly porous matrix composition comprising a matrix carrier and at least one active ingredient dissolved in an oil or mixture of oils as described in the present invention, wherein the oil or mixture of oils alone or in combination with the active ingredient have a specific gravity higher than the specific gravity of water.

Such highly porous materials are known to those skilled in the art, and may include synthetic organic polymers such as nylon beads, polymethyl methacrylates or other porous materials, many of them available commercially; synthetic inorganic porous materials such as silica or silica gels or synthetic zeolites; porous materials of naturals sources such as mica and zeolites.

Generally, the microparticulate highly porous matrix composition can be prepared by adsorbing the active ingredients on a highly porous material either directly, as described in example 19 of the present invention, or by dissolving the active ingredients and the oil in a volatile organic solvent, then adsorbing on the solid (highly porous material) and evaporating the organic solvent. In the highly porous matrix composition, the active ingredient is preferably dissolved in the oil. The active ingredient and oil are embedded in the porous matrix, preferably reside in the matrix pores. As long as the microparticles are kept dry or in a formulation that is mostly water, the oils will reside preferably in the porous matrix.

The oil or at least one oil in the oil mixtures is of the structural formula [I]:

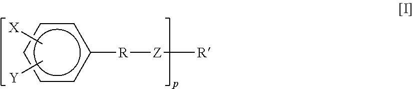

Wherein
R represents (CH₂)ₘ, where m is an integer from 0 to 3,
Z is selected from the group consisting of COO and OOC.
p is an integer from 1 to 3,
R′ is selected from the group consisting of an alkyl, aryl, arylalkyl, alkenyl, alcohol, polyol, benzoyl, benzoyl derivatives and azacyclic esters,
and wherein X and Y are independently selected from the group consisting of hydrogen, alkyl, alkoxy, amine, acetoxy ester, ether, halide, hydroxy, ketone, methylamine, and a nitro group.
   Preferably R′ includes 1 to 50 carbon atoms.
   Preferably X and Y independently include 0 to 10 carbon atoms.
   The oil may be for example, dipropylene glycol dibenzoate (X, Y=H, m=0, Z=COO, R′=(CH₂)₃O(CH₂)₃, p=2), diethylene glycol dibenzoate (X, Y=H, m=0, Z=COO, R′=(CH₂)₂O(CH₂)₂, p=2), methyl benzoate (X, Y=H, m=0, Z=COO, R′=CH₃, p=1), benzyl benzoate (X, Y=H, m=0, Z=COO, R′=CH₂C₆H₆, p=1), ethyl benzoate (X, Y=H, m=0, Z=COO, R′=CH₂CH₃, p=1), phenyl benzoate (X, Y=H, m=0, Z=COO, R′=C₆H₅, p=1), phenyl ethyl benzoate (X, Y=H, m=1, Z=COO, R′=C₆H₅CH₂CH₃, p=1), phenyl salicylate (X=2-OH, Y=H, m=0, Z=COO, R′=C₆H₅, p=1), methyl salicylate (X=2-OH, Y=H, m=0, Z=COO, R′=CH₃, p=1), glycerol tribenzoate (X, Y=H, m=0, Z=COO, R′=(CH₂)₃, p=3), isopropyl benzoate (X, Y=H, m=0, Z=COO, R′=CH(CH₃)₂, p=1), propyl benzoate (X, Y=H, m=0, Z=COO, R′=(CH₂)₂CH₃, p=1), ethyl salicylate (X=2-OH, Y=H, m=0, Z=COO, R′=C₂H₅, p=1), Ethyl Anisate (X=4-OCH₃, Y=H, m=0, Z=COO, R′=CH₂CH₃, p=1), benzyl propionate (X, Y=H, m=1, Z=OOC, R′=CH₂CH₃, p=1), anisyl acetate (X=CH₃O, Y=H, m=1, Z=OOC, R′=CH₃, p=1), phenyl ethyl acetate (X=CH₃CH₂, Y=H, m=0, Z=OOC, R′=CH₃, p=1) or mixtures thereof.

In a preferred embodiment, the oil is dipropylene glycol dibenzoate or a mixture of dipropylene glycol dibenzoate with ethyl benzoate and ethyl salicylate. In another preferred embodiment the oil is methyl salicylate or ethyl salicylate.

When a mixture including dipropylene glycol dibenzoate, ethyl salicylate and ethyl benzoate is used, the weight fraction of dipropylene glycol dibenzoate is from about 31 to about 88.5; the weight fraction of ethyl salicylate is from about 7 to about 42; the weight fraction of ethyl benzoate is from about 4.5 to about 27.

Most preferably the weight fractions of dipropylene glycol dibenzoate: ethyl salicylate: ethyl benzoate are 77:14:9 respectively.

In yet another preferred embodiment the oil is selected from the group consisting of ethyl benzoate, benzyl proprionate, anisyl acetate, phenyl ethyl benzoate, phenyl ethyl acetate, ethyl anisate and mixtures thereof.

The microparticulate composition of the present invention may comprise a mixture of oils including:
(a) at least one oil having a specific gravity lower than the specific gravity of water;
(b) at least one oil having a specific gravity higher than the specific gravity of water;
   wherein the mixture of oils have a specific gravity higher than the specific gravity of water.

The microparticulate compositions of the present invention may include for example a mixture of oils comprising:
(a) at least one oil selected from the group consisting of C12-C15 alkyl benzoate, isostraryl benzoate, PPG-15 stearyl ether benzoate, octyldodecyl benzoate, stearyl benzoate, methyl gluceth-20 benzoate, poloxamer 182 dibenzoate, poloxamer 105 benzoate, transcutol, dioctyl malate, diethylhexylmaleate, diethylhexylsebacate, diethylhexyladipate, diisopropyladipate, diisopropylsebacate, diisopropylmaleate, ethylhexylsalicylate, tridecylsalicylate, butyloctylsalycilate, isopropylmyristate and mixture thereof;
(b) at least one oil at as defined in the present invention by formula (I)

wherein the mixture of oils alone or in combination with the active ingredient have a specific gravity higher than the specific gravity of water.

The active ingredient in the microcapsular or microparticulate compositions may be for example a suncscreen agent, a dermatological agent, a pharmaceutical agent, a vitamin, an anti-inflammatory agent, an analgesic, an anti-fungal agent, an anti-biotic, an anti-viral agent, an anti-acne agent, an anti histamine, an enzyme, a co-enzyme, a humectant, a dermatological agent, an insect repellent, a perfume, a color, a dye, a skin whitening agent, an aromatic oil, a flavoring agent, a dental agent, an anti-parasitic agent, a muscle relaxant, a steroid, a hormone, an astringent, a toner, an anti-oxidant, or mixtures thereof.

In one preferred embodiment of the current invention the active is a sunscreen agent.

The sunscreen agent may be for example a UVA absorber, a UVB absorber or a combination thereof.

The sunscreen agent may be for example, benzophenones, 4,4′-methoxy-t-butyldibenzoylmethane, 4-isopropyl dibenzoylmethane, 4-methylbenzylidene camphor, 3-benzylidene camphor, Polyacrylamidomethyl benzylidene camphor.

The benzophenones are preferably oxybenzone (benzophenone-3), benzophenone-1, benzophenone-2, benzophenone-6 and benzophenone-8. Preferably the dermatological agent is a peroxide.

The peroxides include but not limited to peroxyacids of carboxylic acids, peroxyesters of carboxylic acids and the dimeric product of carboxylic peroxyacids. Exemplary peroxides include t-butyl peroxyesters of straight and branched chain aliphatic carboxylic acids, and dimeric peroxides such as lauroyl peroxide and benzoyl peroxide. In addition the peroxides include urea peroxide. A preferred peroxide for use in the present invention is benzoyl peroxide and urea peroxide.

The active ingredient may be dissolved in the oil or mixtures of oils at a concentration of about 0.1 to about 50 wt. %, preferably at a concentration of about 2 to about 40%, more preferably at a concentration of about 5 to about 35%.

Attaining high concentrations of actives dissolved in the carrier oils is important when high loadings of actives in microcapsular or microparticulate systems are used. Such non-homogeneous systems have many advantages in pharmaceutical and cosmetic preparations, as they facilitate control of properties of the formulation such as isolation of actives, control of release or prevention of release of certain actives, stabilization of sensitive actives etc, by attaining micro-domains in the formula. In many cases, to make these systems viable, the microcapsular or microparticular system must contain a high loading of the active (up to 80-90 wt. %) to allow mixing with other components of the formula while still achieving a sufficiently high concentration of the active in the final preparation.

In case the active ingredient is encapsulated in a microcapsular composition, the microcapsular composition may be dispersed in a pharmaceutical or cosmetic composition for example an oil, ointment, cream or gel. The pharmaceutical or cosmetic composition may further comprise at least one active ingredient not encapsulated within the microcapsular composition.

Alternatively, the active ingredient may be embedded in a microparticulate composition, the microparticulate composition may be dispersed in a pharmaceutical or cosmetic composition for example an oil, ointment, cream or gel. The pharmaceutical or cosmetic composition may further comprise at least one active ingredient not embedded in the microparticulate composition.

The present invention further provides a process for preparing a pharmaceutical or cosmetic composition as described in the present invention comprising at least the step of dissolving an active ingredient in an oil or mixture of oils, wherein the oil or mixture of oils alone or in combination with the active ingredient have a specific gravity higher than the specific gravity of water.

In this process, the oils and active ingredients used for preparing the pharmaceutical or cosmetic compositions are the same as described and detailed above.

In case a microcapsular composition is to be prepared, the process for preparing a microcapsular composition comprises the steps of:
(a) dissolving an active ingredient in a mixture including:
   (i) an oil or mixture of oils, wherein the oil or mixture of oils alone or in combination with the active ingredient have a specific gravity higher than the specific gravity of water; and
   (ii) sol-gel precursors
   to form a hydrophobic solution;
(b) emulsifying the hydrophobic solution in an aqueous solution under high shear forces;
(c) mixing and stirring the emulsion obtained in step (b) with a second aqueous solution at a predetermined pH to obtain the microcapsules; and
(d) isolating the obtained microcapsules through precipitation or sedimentation in a centrifuge.

In this process, the oils and active ingredients used for preparing the microcapsular compositions are the same as described and detailed above.

Preferably the pH in step (c) is from about 2 to about 13 more preferably from about 2 to about 4. In another preferred embodiment the pH is from about 8.5 to about 11.5.

The sol-gel precursors may be for example a metal or semi-metal alkoxide monomers, metal ester monomers, semi-metal ester monomers or monomers of the formula M(R)ₙ(P)ₘ, wherein M is a metallic or semi metallic element, R is a hydrolyzable substituent, n is an integer from 2 to 6, P is a non polymerizable substituent and m is an integer from 0 to 6; or partially hydrolyzed and partially condensed polymer thereof, or any mixture thereof.

The use of an oil or mixtures of oils having a specific gravity higher than water or an oil or mixture of oils in combination with at least one active ingredient wherein said combination is characterized by having a specific gravity higher than the specific gravity of water, is particularly advantageous in the preparation of microcapsules since the microcapsules formed can be easily isolated due to their specific gravity higher than water. This enables isolating the obtained microcapsules through precipitation or sedimentation in a centrifuge, which is an easy, and lower in cost procedure.

The present invention further relates to a method of treating a medical condition in a human patient, the medical condition may be for example a skin, hair, ear, mucosal membrane, rectal, nasal or dental disease, disorder or condition. The method comprises the step of administering a composition as described in the present invention to a patient in need of such treatment.

The condition may be for example acne, psoriasis, seborrea, bacterial infection, viral infection, fungal infection, inflammation, aging signs, dandruff and cavity.

It is to be understood that the invention is not limited in its application to the details of construction and the arrangement of the components set forth in the following description. The invention includes other embodiments and can be practiced or implemented in various ways. Also it is to be understood that the phraseology and terminology employed herein is for the purpose of description only and should not be regarded as limiting.

### EXAMPLES

Reference is now made to the following examples, which together with the above descriptions, illustrate the invention in a non limiting fashion.

### Example 1

### Solubility of BMDBM and Other Solid Sunscreen Actives in Different Solvents, Including DipropyleneGlycol Dibenzoate

The solubility of commonly used sunscreen active ingredients was tested by heating a solution of below-mentioned solvents or their mixture to 50° C. under stirring. Access amount of the sunscreen active was added. After thorough mixing to dissolve the solid, the solution was allowed to cool to 25° C. over night in the water bath under constant oscillation and the access active precipitated. The solution was filtered using 0.2 μm PTFE filter, and the concentration of BMDBM was determined by HPLC equipped with UV diode array detector against standard solution of BMDBM. For the other sunscreen actives, the concentration of the active was determined spectroscopically against standard solution of the active. Table 1 lists the solubility values obtained for BMDBM and other sunscreen actives in different commercially available solvents.

TABLE 1

| | | Solvent | Solvent specific | | |
|---|---|---|---|---|---|
| | Solvent name, | manufacturer, | gravity, acc. | Solubility | |
| # | CAS #[XXXXX] | Solvent trade name | to D25/25 | 25° C. | 8° C. |
| | | | | BMDBM | |
| | | | | concentration, wt % | |
| 1 | Dipropylene glycol Dibenzoate | Finetex | 1.12 | 23.35 | 21.72 |
| | [27138-31-4] | Finsolv PG-22 | | | |
| 2 | Dipropylene glycol Dibenzoate | ALZO International | 1.117 | 26.66 | 17.7 |
| | [27138-31-4] | Dermol DPG-2B | | | |
| 3 | Ethyl benzoate | Haarmann & Reimer's | 1.043-1.046 | 40.45 | — |
| | [93-89-0] | | | | |
| 4 | Ethyl salicylate | Haarmann & Reimer's | 1.126-1.130 | 34.74 | — |
| | [118-61-6] | | | | |
| 5 | Benzyl propionate | Haarmann & Reimer's | 1.028-1.032 | 28.49 | — |
| | [122-63-4] | | | | |
| 6 | DermolDPG-2B/Ethyl | As mentioned above | 1.111* | 29.78 | 29.78 |
| | salicylat/Ethylbenzoate = | | | | |
| | 77/14/9(w/w) | | | | |
| | | | | Methyl benzyledene | |
| | | | | camphor | |
| | | | | concentration, wt % | |
| 7 | Dipropylene glycol Dibenzoate | Finetex | 1.12 | 30.16 | — |
| | [27138-31-4] | Finsolv PG-22 | | | |
| | | | | Benzophenone-3 | |
| 8 | Dipropylene glycol Dibenzoate | Finetex | 1.12 | 26.6 | — |
| | [27138-31-4] | Finsolv PG-22 | | | |

*this value was calculated theoretically, according to the following equation: 1/ρ = 0.77/1.117 + 0.14/1.128 + 0.09/1.0445, and finally ρ = 1.111

The results show that the solubility of BMDBM in Dipropelene glycol Dibenzoate (solvent #1) is not significantly changed at 25° C. compared to 8° C. and is identical (at 25° C. compared to 8° C.) in a specific combination of solvents including Dermol DPG/Ethyl Salicylate/Ethylbenzoate (mixture #6). Therefore, this combination of solvents (mixture #6) is highly advantageous since it enables the production of a stable composition, which is not affected by temperature changes during its production.

### Example 2

### Encapsulation of BMDBM Dissolved in Mixture #6 (from Table 1 in Example 1) in Silica Microcapsules

441 g BMDBM was completely dissolved in 1134 g of the mixture #6 (from table 1) and 675 g of TEOS was added. The organic phase was emulsified in 2250 g 1.6% CTAC in water under high shear. The vessel walls were cooled by immersion in an ice-water bath during the homogenizing process. This emulsion was then poured into a 20 L PP reactor, equipped with upper stirrer, containing 8148 g HCL aqueous solution at pH 3.8. The solution was stirred at 60 rpm.

After 7 days the product was precipitated in a centrifuge, and the cake was washed, and re-suspended in a solution containing PVP 0.8%, 12 mM Citrate buffer at pH 4. This procedure resulted in the production of silica particles with mean particle size of 1.37 μm.

### Example 3

### Encapsulation of BMDBM Dissolved in Mixture #6 (from Table 1 in Example 1) in Silica Microcapsules

122 g BMDBM was completely dissolved in 313 g of the mixture #6 (from table 1) and 435 g of TEOS was added. The organic phase was emulsified in 3630 g 1% CTAC in water under high shear. The vessel walls were cooled by immersion in an ice-water bath during the homogenizing process. This emulsion was then poured into a 20 L PP reactor, equipped with upper stirrer, containing 3630 g HCL aqueous solution at pH 3.8. The solution was stirred at 60 rpm.

After 7 days the product was precipitated in a centrifuge, and the cake was washed, and re-suspended in a solution containing PVP 0.8%, 12 mM Citrate buffer at pH4. This procedure resulted in the production of silica particles with mean particle size of 1.28 μm.

### Example 4

### Solubility of Benzoyl Peroxide in Finsolve PG-22

The solubility of benzoyl peroxide in dipropylene glycol dibenzoate (Finsolv PG 22) was measured in a method similar to Example 1. Benzoyl peroxide concentration was determined by titration according to USP. The solubility of benzoyl peroxide in Finsolve PG-22 is 6 wt. %.

### Example 5

### Density of Mixtures of Finsolv PG-22 with Finsolv TN

The density of Finsolv TN (C12-C15 alkyl benzoate) is lower than that of pure water. Consequently, this solvent which is commonly used in cosmetic products, can not be utilized when precipitation processes are to be performed especially when the density (specific gravity) of the water phase (synthesis medium) of the microcapsules or microparticles is higher than the density of Finsolve TN. The density of binary mixtures of Finsolv PG-22 and Finsolv TN was measured. The results are given in Table 2.

TABLE 2

| Finsolv PG-22 | Finsolv TN | Density at r.t.⁽*⁾ |
|---|---|---|
| Wt. % | wt. % | g/mL |
| 20 | 80 | 0.98 |
| 40 | 60 | 1.01 |
| 50 | 50 | 1.04 |
| 70 | 30 | 1.06 |

⁽*⁾r.t. = room temperature

Although the density (specific gravity) of Finsolv TN (C12-C15 alkyl benzoate) is approximately 0.92, which is lower than that of pure water, a combination of Finsolv PG-22 with Finsolv TN increases the density above that of Finsolv TN, with increasing the concentration of Finsolv PG-22 in the mixture. Therefore a mixture of Finsolv TN and Finsolv PG-22 is advantageous when there is a need to increase the density of the oils above that of the water phase to enable precipitation.

### Example 6

### Encapsulation of BMDBM and Finsolv PG-22 in Silica Microcapsules

18 g BMDBM was mixed with 42 g dipropylene glycol dibenzoate (Finsolv PG 22) and 40 g tetraethoxy silane (TEOS). The organic phase was emulsified in 400 g 1% cetyltrimethyl ammonium chloride (CTAC) under high shear. The vessel walls were cooled by immersion in an ice-water bath during the homogenizing process. This emulsion was then poured into an IKA LR-A 1000 Laboratory reactor, equipped with Eurostar Power control −visc P4 stirrer, containing 400 g NaOH aqueous solution at pH 10. The solution is stirred at 200 rpm. After 7 days the product was precipitated in a centrifuge, and the cake was washed, and re-suspended in a solution containing PVP 0.08%, 12 mM Citrate buffer, pH 4. This procedure resulted in the production of silica particles of 0.5 to 10μ in size, with a mean particle size of 0.8μ.

### Example 7

### Encapsulation of BPO and Finsolve PG 22 in Silica

60 grams of 7% (w/w) benzoyl peroxide (BPO) in dipropyleneglycol dibenzoate were mixed with 40 grams of TEOS. The organic phase was emulsified in 400 grams of an aqueous solution containing 1 CTAC under high shear forces. The vessel walls were cooled by immersion in an ice-water bath during the homogenization process. This emulsion was then poured into an IKA LR-A 1000 Laboratory reactor, equipped with Eurostar Power control-visc P4 stirrer, containing 400 grams NaOH aqueous solution at pH 10. The solution was stirred at 200 rpm. After 3 days the product was separated by centrifuge and washed. The final product was resuspended in water with a final concentration of 4-6% BPO encapsulated within silica particles of 0.5-5 microns.

### Example 8

### Encapsulation of BPO and Schercemol GMIS in Silica

Capsules of silica containing BPO dissolved in oil were made in a similar manner to example 7, using mixtures of dipropylene glycol dibenzoate with clycerylisostearate (98:2). Silica particles encapsulating BPO, of 0.3 to 3 micron were obtained.

### Example 9

### Encapsulation of BPO and Finsolv PG 22 and Montane 80 in Silica

Capsules of silica containing BPO dissolved in oil were made in a similar manner to example 7, using mixtures of dipropylene glycol dibenzoate with sorbitan monooleate (98:2). Silica particles encapsulating BPO, of 0.3 to 3 micron were obtained.

### Example 10

### Encapsulation of BPO and Anisyl Acetate and Montane 80 in Silica

Capsules of silica containing 10 wt % BPO dissolved in oil were made in a similar manner to example 7, using mixtures of Anisyl acetate with sorbitan monooleate (98:2). In the present example CTAC was replaced by Tween 80 in the water phase. Silica particles encapsulating BPO, of 0.3 to 3 micron were obtained.

### Example 11

### Encapsulation of BPO and Benzyl Propionate and Montane 80 in Silica

Capsules of silica containing 10 wt % BPO dissolved in oil were made in a similar manner to example 10, using mixtures of Benzyl Propionate with sorbitan monooleate (98:2). Silica particles encapsulating BPO, of 0.3 to 3 micron were obtained.

### Example 12

### Encapsulation of BPO and Ethyl Benzoate and Montane 80 in Silica

Capsules of silica containing 10 wt % BPO dissolved in oil were made in a similar manner to example 10, using mixtures of Ethyl Benzoate with sorbitan monooleate (98:2). Silica particles encapsulating BPO, of 0.3 to 3 micron were obtained.

### Example 13

### Encapsulation of BPO and Phenyl Ethyl Benzoate and Montane 80 in Silica

Capsules of silica containing 10 wt % BPO dissolved in oil were made in a similar manner to example 10, using mixtures of Ethyl phenyl benzoate with sorbitan monooleate (98:2). Silica particles encapsulating BPO, of 0.3 to 3 micron were obtained.

### Example 14

### Encapsulation of BPO and Phenyl Ethyl Acetate and Montane 80 in Silica

Capsules of silica containing 10 wt % BPO dissolved in oil were made in a similar manner to example 10, using mixtures of phenyl ethyl acetate with sorbitan monooleate (98:2). Silica particles encapsulating BPO, of 0.3 to 3 micron were obtained.

### Example 15

### Encapsulation of BPO and Ethyl Salicylate and Montane 80 in Silica

Capsules of silica containing 10 wt % BPO dissolved in oil were made in a similar manner to example 10, using mixtures of ethyl salicylate with sorbitan monooleate (98:2). Silica particles encapsulating BPO, of 0.3 to 3 micron were obtained.

### Example 16

### Encapsulation of BPO and Methyl Salicylate and Montane 80 in Silica

Capsules of silica containing 10 wt % BPO dissolved in oil were made in a similar manner to example 10, using mixtures of methyl salicylate with sorbitan monooleate (98:2). Silica particles encapsulating BPO, of 0.3 to 3 micron were obtained.

### Example 17

### Encapsulation of BPO and Ethyl Anisate and Montane 80 in Silica

Capsules of silica containing 10 wt % BPO dissolved in oil were made in a similar manner to example 10, using mixtures of Ethyl Anisate with sorbitan monooleate (98:2). Silica particles encapsulating BPO, of 0.3 to 3 micron were obtained.

### Example 18

### Sunscreen Composition Including BMDBM and Finsolv PG22

| | | | | | | |
|---|---|---|---|---|---|---|
| Formulation No. | 51 | 52 | 54 | 55 | 56 | 57 |

Phase A

| | | | | | | |
|---|---|---|---|---|---|---|
| Abil EM 90 | 6 | 6 | 6 | 6 | 6 | 6 |
| Arlamol HD | 23 | 23 | 23 | 5.4 | 23 | 22 |
| Cetiol SN | 19.5 | 19.5 | 19.5 | 19.5 | 19.5 | 2.9 |
| Vitamin E Acetat | 2 | 2 | 2 | 2 | 2 | 2 |
| OMC | — | — | — | 17.6 | — | 17.6 |
| BMDBM/PG-22 | — | — | — | — | 20 | 20 |

Phase B

| | | | | | | |
|---|---|---|---|---|---|---|
| NaCl | 2 | 2 | 2 | 2 | 2 | 2 |
| Glycerin | 8 | 8 | 8 | 8 | 8 | 8 |
| Pantenol | 2 | 2 | 2 | 2 | 2 | 2 |
| UV Pearls OMC | 50 | — | 50 | — | — | — |
| UV Pearls | — | 40 | 40 | 40 | — | — |
| BMDBM/PG-22 | | | | | | |
| Water | 87.5 | 97.5 | 47.5 | 97.5 | 117.5 | 117.5 |

Preparation: heat up phase A to 80° C. whilst stirring, cool down to 40° C. and add phase B (cold) to phase A whilst stirring.

### Homogenize for 1 min.

### Examples 19

### Sunscreen Composition Including Adsorbed BMDBM and Finsolv PG22

| | |
|---|---|
| Trade name (INCI/CTFA name) | wt. % |

Phase A

| | |
|---|---|
| Squalane | 5.00 |
| Cetyl Alcohol | 1.50 |
| Glyceyl stearate and PEG-100 stearate | 5.00 |
| Propyl parabene | 0.10 |
| Euxyl K-100 | 0.07 |

Phase B

| | |
|---|---|
| Methyl parabene | 0.20 |
| Imidazolydene urea | 0.30 |
| EDTA | 0.05 |
| Water | 64.0 |

Phase C

| | |
|---|---|
| Butyl methoxydibenzoylmethane | 1.80 |
| Dipropylenglycol dibenzoate | 3.60 |
| Silica | 6.00 |
| Water | 12.0 |

Preparation: heat up phase A to 75° C. whilst stirring, heat phase B to 80° C. whilst stirring. Add phase B to A, homogenize for 5 minutes and cool down. Dissolve BMDBM in dipropyleneglycol dibenzoate, add drop wise to the silica powder whilst stirring to obtain dry looking powder. Mix with water whilst stirring to obtain phase C. Mix phase C with the emulsion of phase A and B (cooled), homogenize briefly.

### Example 20

### Water Based Gel for Treatment of Acne Containing BPO and Finsolv PG 22

| | |
|---|---|
| Trade name (INCI/CTFA name) | wt. % |

Phase A

| | |
|---|---|
| Sepigel 305 (Polyacrylamide/C13.14 | 2.00% |
| Isoparaffin/Laureth-7) | |
| LANOL 1688 (Cetearyl octanoate) | 10.00% |

Phase B

| | |
|---|---|
| Encapsulated BPO dispersion in | 50.00% |
| water (5% w/w) | |
| Water | qs 100% |

Preparation: mix A and B separately at room temperature, then add phase B to A with moderate stirring.

### CONCLUSIONS

All the above compositions, as described in the above examples, were found to be simplified in production. Dipropylene glycol dibenzoate was found to be a good solvent for BPO. This property of dipropylene glycol dibenzoate supports its use as a solubilizer for the pharmaceutical agent, to obtain a non-irritant BPO preparation for the treatment of Acne. The use of an oil or mixture of oils having a specific gravity higher than the specific gravity of water as described in the above examples is highly advantageous especially in the preparation of microcapsules since the microcapsules can be easily isolated through precipitation or sedimentation in a centrifuge, it enables high loading of the active ingredient within the microcapsules, the active ingredient does not crystallize within the microcapsules core and it enables the production of a stable product with a uniform microcapsules' size of 0.3 to 3 microns.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A microcapsular composition comprising:
(a) a core, including at least one active ingredient in combination with an oil or mixture of oils, wherein said oil or mixture of oils alone or in combination with said active ingredient have a specific gravity higher than the specific gravity of water; and
(b) at least one microcapsular shell encapsulating said core.

2. The microcapsular composition of claim 1 wherein said oil or at least one oil in the oil mixtures is of the structural formula [I]: 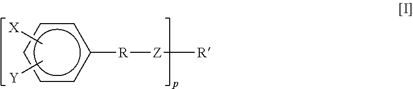
Wherein
R represents (CH₂)ₘ, where m is an integer from 0 to 3,
Z is selected from the group consisting of COO and OOC.
p is an integer from 1 to 3,
R′ is selected from the group consisting of an alkyl, aryl, arylalkyl, alkenyl, alcohol, polyol, benzoyl, benzoyl derivatives and azacyclic esters,
and wherein X and Y are independently selected from the group consisting of hydrogen, alkyl, alkoxy, amine, acetoxy ester, ether, halide, hydroxy, ketone, methylamine, and a nitro group.

3. The microcapsular composition of claim 2, wherein said R′ includes 1 to 50 carbon atoms.

4. The microcapsular composition of claim 2, wherein said X and said Y independently include 0 to 10 carbon atoms.

5. The microcapsular composition of claim 2 wherein said oil of formula [I] as defined by X, Y, m, Z, R′ and p is selected from the group consisting of:
X, Y=H, m=0, Z=COO, R′ (CH₂)₃O(CH₂)₃,
X, Y=H, m=0, Z=COO, R′=(CH₂)₂O(CH₂)₂, p=2;
X, Y=H, m=0, Z=COO, R′=CH₃, p=1;
X, Y=H, m=0, Z=COO, R′=CH₂C₆H₆, p=1;
X, Y=H, m=0, Z=COO, R′=CH₂CH₃, p=1;
X, Y=H, m=0, R′=C₆H₅, p=1;
X, Y=H, m=1, Z=COO, R′=C₆H₅CH₂CH₃,
X=2-OH, Y=H, m=0, Z=COO, R′=C₆H₅, p=1;
X=2-OH, Y=H, m=0, Z=COO, R′=CH₃, p=1;
X, Y=H, m=0, Z=COO, R′=(CH₂)₃, p=3;
X, Y=H, m=0, Z=COO, R′=CH(CH₃)₂, p=1;
X, Y=H, m=0, R′=(CH₂)₂CH₃, p=1;
X=2-OH, Y=H, m=0, Z=COO, R′=CH₂H₅, p=1;
X=4-OCH₃, Y=H, m=0, Z=COO, R′=CH₂CH₃, p=1;
X, Y=H, m=1, Z=OOC, R′=CH₂CH₃, p=1;
X=CH₃O, Y=H, m=1, Z=OOC, R′=CH₃, p=1;
X=CH₃CH₂, Y=H, m=0, Z=OOC, R′=CH₃, p=1;
and mixtures thereof.

6. The microcapsular composition of claim 1 wherein said mixture of oils comprises
(a) at least one oil having a specific gravity lower than the specific gravity of water;
(b) at least one oil having a specific gravity higher than the specific gravity of water;
wherein said mixture of oils alone or in combination with the active ingredient of claim 1 have a specific gravity higher than the specific gravity of water.

7. The microcapsular composition of claim 1 wherein said mixture of oils comprises
(a) at least one oil selected from the group consisting of C12-C15 alkyl benzoate, isostraryl benzoate, PPG-15 stearyl ether benzoate, octyldodecyl benzoate, stearyl benzoate, methyl gluceth-20 benzoate, poloxamer 182 dibenzoate, poloxamer 105 benzoate, transcutol, dioctyl malate, diethylhexylmaleate, diethylhexylsebacate, diethylhexyladipate, diisopropyladipate, diisopropylsebacate, diisopropylmaleate, ethylhexylsalicylate, tridecylsalicylate, butyloctylsalycilate, isopropylmyristate and mixture thereof;
(b) at least one oil as defined in claim 2 or 5;
wherein said mixture of oils alone or in combination with the active ingredient of claim 1 have a specific gravity higher than the specific gravity of water.

8. The composition of claim 1 wherein said oil is a mixture of dipropylene glycol dibenzoate, ethyl salicylate and ethylbenzoate.

9. The microcapsular composition of claim 1 wherein said composition is in the form of sol-gel microcapsules.

10. The microcapsular composition of claim 1, wherein said active ingredient is dissolved in said oil or mixtures of oils at a concentration of about 0.1 to about 50 wt. %.

11. The microcapsular composition of claim 1, wherein said active ingredient is dissolved in said oil or mixtures of oils at a concentration of about 2 to about 40 wt. %.

12. The microcapsular composition of claim 1 wherein said active ingredient is selected from the group consisting of a suncscreen agent, a dermatological agent, a pharmaceutical agent, a vitamin, an anti-inflammatory agent, an analgesic, an anti-fungal agent, an anti-biotic, an anti-viral agent, an anti-acne agent, an anti histamine, an enzyme, a co-enzyme, a humectant, a dermatological agent, an insect repellent, a perfume, a color, a dye, a skin whitening agent, an aromatic oil, a flavoring agent, a dental agent, an anti-parasitic agent, a muscle relaxant, a steroid, a hormone, an astringent, a toner, an anti-oxidant, and mixtures thereof.

13. The microcapsular composition of claim 1, wherein said active ingredient is a sunscreen agent.

14. The microcapsular composition of claim 13 wherein said sunscreen agent is selected from the group consisting of a UVA absorber, a UVB absorber or a combination thereof.

15. The microcapsular composition of claim 13 wherein said sunscreen agent is selected from the group consisting of benzophenones, 4,4′-methoxy-t-butyldibenzoylmethane, 4-isopropyl dibenzoylmethane, 4-methylbenzylidene camphor, 3-benzylidene camphor, polyacrylamidomethyl benzylidene camphor and mixtures thereof.

16. The microcapsular composition of claim 15 wherein said benzophenones are selected from the group consisting of benzophenone-3, benzophenone-1, benzophenone-2, benzophenone-6 and benzophenone-8 and mixtures thereof.

17. The microcapsular composition of claim 12 wherein said dermatological agent is a peroxide.

18. The microcapsular composition of claim 17 wherein said peroxide is selected from the group consisting of benzoyl peroxide, urea peroxide and mixtures thereof.

19. The microcapsular composition of claim 1 wherein said active ingredient is benzoyl peroxide and said oil is dipropyleneglycol dibenzoate.

20. The microcapsular composition of claim 1, wherein the microcapsules are dispersed in a pharmaceutical or a cosmetic composition.

21. A microparticulate highly porous matrix composition comprising a matrix carrier and at least one active ingredient dissolved in an oil or mixture of oils, wherein said oil or mixture of oils alone or in combination with said active ingredient have a specific gravity higher than the specific gravity of water.

22. The microparticulate composition of claim 21 wherein said oil or at least one oil in the oil mixtures is of the structural formula [I]: 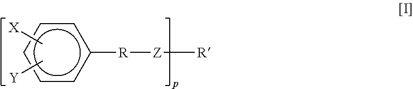
Wherein
R represents (CH₂)ₘ, where m is an integer from 0 to 3,
Z is selected from the group consisting of COO and OOC.
p is an integer from 1 to 3,
R′ is selected from the group consisting of an alkyl, aryl, arylalkyl, alkenyl, alcohol, polyol, benzoyl, benzoyl derivatives and azacyclic esters,
and wherein X and Y are independently selected from the group consisting of hydrogen, alkyl, alkoxy, amine, acetoxy ester, ether, halide, hydroxy, ketone, methylamine, and a nitro group.

23. The microparticulate composition of claim 22, wherein said R′ includes 1 to 50 carbon atoms.

24. The microparticulate composition of claim 22, wherein said X and said Y independently include 0 to 10 carbon atoms.

25. The microparticulate composition of claim 22 wherein said oil of formula [I] as defined by X, Y, m, Z, R′ and p is selected from the group consisting of:
X, Y=H, m=0, Z=COO, R′=(CH₂)₃O(CH₂)₃, p=2;
X, Y=H, m=0, Z=COO, R′=(CH₂)₂O(CH₂)₂, p=2;
X, Y=H, m=0, Z=COO, R′=CH₃, p=1;
X, Y=H, m=0, Z=COO, R′=CH₂C₆H₆, p=1;
X, Y=H, m=0, Z=COO, R′=CH₂CH₃, p=1;
X, Y=H, m=0, Z=COO, R′=C₆H₅, p=1;
X, Y=H, m=1, Z=COO, R′=C₆H₅CH₂CH₃, p=1;
X=2-OH, Y=H, m=0, Z=COO, R′=C₆H₅, p=1;
X=2-OH, Y=H, R′=CH₃, p=1;
X, Y=H, m=0, Z=COO, R′=(CH₂)₃, p=3;
X, Y=H, m=0, Z=COO, R′=CH(CH₃)₂, p=1;
X, Y=H, m=0, Z=COO, R′=(CH₂)₂CH₃, p=1;
X=2-OH, Y=H, m=0, Z=COO, R′=CH₂H₅, p=1;
X=4-OCH₃, Y=H, m=0, Z=COO, R′=CH₂CH₃, p=1;
X, Y=H, m=1, Z=OOC, R′=CH₂CH₃, p=1;
X=CH₃O, Y=H, m=1, Z=OOC, R′=CH₃, p=1;
X=CH₃CH₂, Y=H, m=0, Z=OOC, R′=CH₃, p=1;
and mixtures thereof.

26. The microparticulate composition of claim 21 wherein said mixture of oils comprises
(a) at least one oil having a specific gravity lower than the specific gravity of water;
(b) at least one oil having a specific gravity higher than the specific gravity of water;
wherein said mixture of oils alone or in combination with the active ingredient of claim 21 have a specific gravity higher than the specific gravity of water.

27. The microparticulate composition of claim 21 wherein said mixture of oils comprises
(a) at least one oil selected from the group consisting of C12-C15 alkyl benzoate, isostraryl benzoate, PPG-15 stearyl ether benzoate, octyldodecyl benzoate, stearyl benzoate, methyl gluceth-20 benzoate, poloxamer 182 dibenzoate, poloxamer 105 benzoate, transcutol, dioctyl malate, diethylhexylmaleate, diethylhexylsebacate, diethylhexyladipate, diisopropyladipate, diisopropylsebacate, diisopropylmaleate, ethylhexylsalicylate, tridecylsalicylate, butyloctylsalycilate, isopropylmyristate and mixture thereof;
(b) at least one oil as defined in claim 22 or 25;
wherein said mixture of oils alone or in combination with the active ingredient of claim 21 have a specific gravity higher than the specific gravity of water.

28. The composition of claim 21 wherein said oil is a mixture of dipropylene glycol dibenzoate, ethyl salicylate and ethylbenzoate.

29. The microparticulate composition of claim 21, wherein said active ingredient is dissolved in said oil or mixtures of oils at a concentration of about 0.1 to about 50 wt. %.

30. The microparticulate composition of claim 21, wherein said active ingredient is dissolved in said oil or mixtures of oils at a concentration of about 2 to about 40 wt. %.

31. The microparticulate composition of claim 21 wherein said active ingredient is selected from the group consisting of a suncscreen agent, a dermatological agent, a pharmaceutical agent, a vitamin, an anti-inflammatory agent, an analgesic, an anti-fungal agent, an anti-biotic, an anti-viral agent, an anti-acne agent, an anti histamine, an enzyme, a co-enzyme, a humectant, a dermatological agent, an insect repellent, a perfume, a color, a dye, a skin whitening agent, an aromatic oil, a flavoring agent, a dental agent, an anti-parasitic agent, a muscle relaxant, a steroid, a hormone, an astringent, a toner, an anti-oxidant, and mixtures thereof.

32. The microparticulate composition of claim 21, wherein said active ingredient is a sunscreen agent.

33. The microparticulate, composition of claim 32 wherein said sunscreen agent is selected from the group consisting of a UVA absorber, a UVB absorber or a combination thereof.

34. The microparticulate composition of claim 32 wherein said sunscreen agent is selected from the group consisting of benzophenones, 4,4′-methoxy-t-butyldibenzoylmethane, 4-isopropyl dibenzoylmethane, 4-methylbenzylidene camphor, 3-benzylidene camphor, polyacrylamidomethyl benzylidene camphor and mixtures thereof.

35. The microparticulate composition of claim 34 wherein said benzophenones are selected from the group consisting of benzophenone-3, benzophenone-1, benzophenone-2, benzophenone-6 and benzophenone-8.

36. The microparticulate composition of claim 31 wherein said dermatological agent is a peroxide.

37. The microparticulate composition of claim 36 wherein said peroxide is selected from the group consisting of benzoyl peroxide, urea peroxide and mixtures thereof.

38. The microparticulate composition of claim 21, wherein the microparticles are dispersed in a pharmaceutical or a cosmetic composition.

39. A pharmaceutical or cosmetic composition comprising at least one active ingredient in combination with a mixture of oils comprising dipropylene glycol dibenzoate, ethyl salicylate and ethylbenzoate, wherein said mixture of oils alone or in combination with said active ingredient have a specific gravity higher than the specific gravity of water.

40. The composition of claim 39 wherein said active ingredient is selected from the group consisting of a sunscreen agent, a dermatological agent, a pharmaceutical agent, a vitamin, an anti-inflammatory agent, an analgesic, an anti-fungal agent, an anti-biotic, an anti-viral agent, an anti-acne agent, an anti histamine, an enzyme, a co-enzyme, a humectant, a dermatological agent, an insect repellent, a perfume, a color, a dye, a skin whitening agent, an aromatic oil, a flavoring agent, a dental agent, an anti-parasitic agent, a muscle relaxant, a steroid, a hormone, an astringent, a toner, an anti-oxidant, and mixtures thereof.

41. The composition of claim 39, wherein said active ingredient is a sunscreen agent.

42. The composition of claim 41 wherein said sunscreen agent is selected from the group consisting of a UVA absorber, a UVB absorber or a combination thereof.

43. The composition of claim 41 wherein said sunscreen agent is selected from the group consisting of benzophenones, 4,4′-methoxy-t-butyldibenzoylmethane, 4-isopropyl dibenzoylmethane, 4-methylbenzylidene camphor, 3-benzylidene camphor, polyacrylamidomethyl benzylidene camphor and mixtures thereof.

44. The composition of claim 43 wherein said benzophenones are selected from the group consisting of benzophenone-3, benzophenone-1, benzophenone-2, benzophenone-6 and benzophenone-8 and mixtures thereof.

45. The composition of claim 40 wherein said dermatological agent is a peroxide.

46. The composition of claim 45 wherein said peroxide is selected from the group consisting of benzoyl peroxide, urea peroxide and mixtures thereof.

47. The composition of claim 39, wherein the final form of said composition is selected from the group consisting of an ointment, a cream, a lotion, a microcapsules' dispersion, a microparticles' dispersion, an oil, a gel, a solid stick, a milk, an aerosol, a spray, a powder, a foam, a mousse, a shampoo, a hair conditioner, a lacquer, a make-up, a soap, a paste, a lipstick, a lipcare product, an eyeshadow, a blusher, a presun or aftersun preparations, a hair colorant, a hair highlighter preparation, an astringent and a cleanser.

48. A process for preparing a microcapsular composition comprising the steps of:
(a) dissolving an active ingredient in a mixture including:
(i) an oil or mixture of oils, wherein said oil or mixture of oils alone or in combination with said active ingredient have a specific gravity higher than the specific gravity of water; and
(ii) sol-gel precursors
to form a hydrophobic solution;
(b) emulsifying said hydrophobic solution in an aqueous solution under high shear forces;
(c) mixing and stirring the emulsion obtained in step (b) with a second aqueous solution at a predetermined pH to obtain the microcapsules; and
(d) isolating the obtained microcapsules through precipitation or sedimentation in a centrifuge.

49. A method of treating a medical condition in a human patient, said medical condition is selected from the group consisting of a skin, hair, ear, mucosal membrane, rectal, nasal or dental disease, disorder or condition, the method comprising the step of administering a composition as defined in claim 1, 21 or 39 to a patient in need of such treatment.

50. The method of claim 49 wherein said condition is selected from the group consisting of acne, psoriasis, seborrea, bacterial infection, viral infection, fungal infection, inflammation, aging signs, dandruff and cavity.
